SEQUENCE LISTING (1 sequence)

SEQ ID NO: 1 - DNA, 25 bp, homo sapiens
atcatcgtagctggtcagtgtatcc

(19) United States
(12) Patent Application Publication
(10) Pub. No.: US 2005/0095598 A1
(43) Pub. Date: May 5, 2005

(54) Nucleic acid arrays comprising depurination probe features and methods for using the same

(76) Inventors: Paul K. Wolber, Los Altos, CA (US); Eric M. Leproust, San Jose, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/699,281

(22) Filed: Oct. 30, 2003

(51) Int. Cl. C12Q 1/68

(52) U.S. Cl. 435/6

(57) ABSTRACT

In situ produced nucleic acid arrays that include at least one depurination probe feature are provided, where the at least one depurination probe feature is made up of in situ produced depurination probes. In using the subject arrays, the arrays are contacted with a nucleic acid sample that includes a target which specifically binds to the full length depurination probe of the depurination feature, and the amount of resultant duplex nucleic acids in the feature is determined (e.g., based on detected signal from the feature) to evaluate the extent of depurination that occurred during in situ synthesis of the array. The subject arrays find use in a variety of different applications, including array fabrication quality control applications, e.g., to determine the extent of depurination in a given lot of nucleic acid arrays produced using an in situ fabrication protocol. Also provided are computer programming, devices that include the same and kits that find use in practicing the subject methods.

### FIELD OF THE INVENTION

The present invention relates to biopolymeric arrays, particularly in situ produced nucleic acid arrays, and more particularly the quality assessment thereof.

### BACKGROUND OF THE INVENTION

Array assays between surface bound binding agents or probes and target molecules in solution may be used to detect the presence of particular biopolymeric analytes in the solution. The surface-bound probes may be oligonucleotides, peptides, polypeptides, proteins, antibodies or other molecules capable of binding with target biomolecules in the solution. Such binding interactions are the basis for many of the methods and devices used in a variety of different fields, e.g., genomics (in sequencing by hybridization, SNP detection, differential gene expression analysis, identification of novel genes, gene mapping, finger printing, etc.) and proteomics.

One typical array assay method involves biopolymeric probes immobilized in an array on a substrate such as a glass substrate or the like. A solution containing target molecules (“targets”) that bind with the attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern, i.e., a binding complex pattern, on the surface of the substrate which is detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

Biopolymer arrays can be fabricated using either deposition of the previously obtained biopolymers or in situ synthesis methods. The deposition methods basically involve depositing biopolymers at predetermined locations on a substrate that is suitably activated such that the biopolymers can link thereto. Biopolymers of different sequence may be deposited at difference regions on the substrate to yield the completed array. Typical procedures known in the art for deposition of previously obtained polynucleotides, particularly DNA, such as whole oligomers or cDNA, are to load a small volume of DNA in solution in one or more drop dispensers such as the tip of a pin or in an open capillary and, touch the pin or capillary to the surface of the substrate. Such a procedure is described in U.S. Pat. No. 5,807,522. When the fluid touches the surface, some of the fluid is transferred. The pin or capillary must be washed prior to picking up the next type of DNA for spotting onto the array. This process is repeated for many different sequences and, eventually, the desired array is formed. Alternatively, the DNA can be loaded into a drop dispenser in the form of a pulse jet head and fired onto the substrate. Such a technique has been described in WO 95/25116 and WO 98/41531, and elsewhere.

The in situ synthesis methods include those described in U.S. Pat. No. 5,449,754 for synthesizing peptide arrays, as well as WO 98/41531 and the references cited therein for synthesizing polynucleotides (specifically, DNA) using phosphoramidite or other chemistry. Additional patents describing in situ nucleic acid array synthesis protocols and devices include U.S. Pat. Nos. 6,451,998; 6,446,682; 6,440,669; 6,420,180; 6,372,483; 6,323,043; and 6,242,266; the disclosures of which patents are herein incorporated by reference.

Such in situ synthesis methods can be basically regarded as iterating the sequence of depositing droplets of: (a) a protected monomer onto predetermined locations on a substrate to link with either a suitably activated substrate surface (or with previously deposited deprotected monomer); (b) deprotecting the deposited monomer so that it can react with a subsequently deposited protected monomer; and (c) depositing another protected monomer for linking. Different monomers may be deposited at different regions on the substrate during any one cycle so that the different regions of the completed array will carry the different biopolymer sequences as desired in the completed array. One or more intermediate further steps may be required in each iteration, such as oxidation and washing steps.

With respect to in situ preparation of nucleic acid arrays, in many currently employed protocols successive layers are built up, 3′ to 5′, by pulse-jet depositing an appropriate nucleotide phosphoramidite and an activator to each array feature location of a substrate surface, e.g., a glass wafer surface. The substrate is then removed to a flow cell, and the other phosphoramidite cycle steps (e.g., oxidation and deprotection of the 5′-hydroxyl group) are performed in parallel. The substrate is then re-registered, and the next layer is printed.

The synthesis protocol used to fabricate an array of biopolymeric probes can have a significant impact on the functional nature of the in situ synthesized probes and features thereof on the array. For example, the particular probe synthesis protocol employed can have an impact on the percentage of full length probes that are produced in a given feature. In other words, a given in situ synthesis protocol may produce, in addition to full length probe sequences, non-full length sequences, which non-full length sequences can adversely impact the functionality of the feature.

One reason that non-full length sequences may be produced, in addition to desired full length sequences, in a given feature of an array is that in situ produced oligonucleotides are susceptible to depurination side reactions, specifically acid-catalyzed depurination, shown in below in Scheme 1. 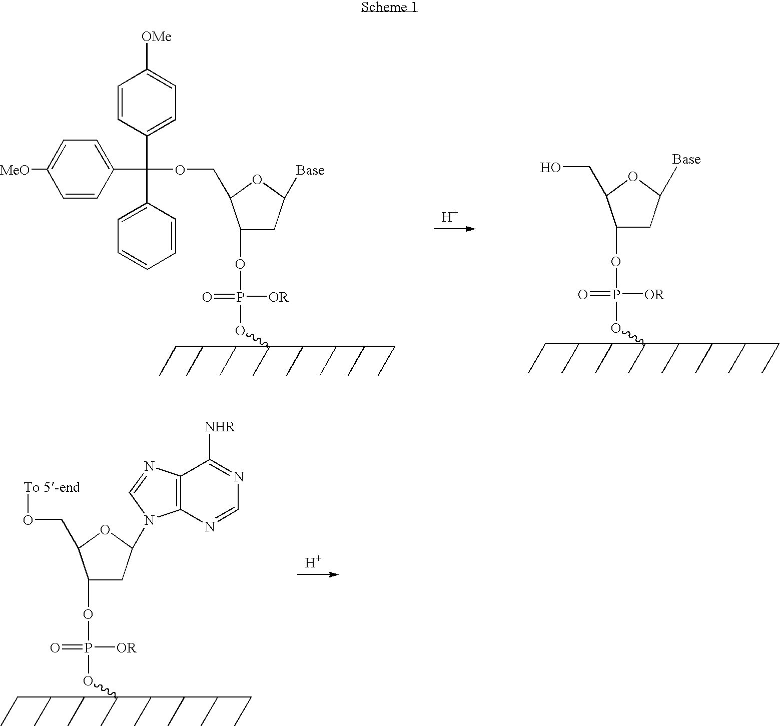 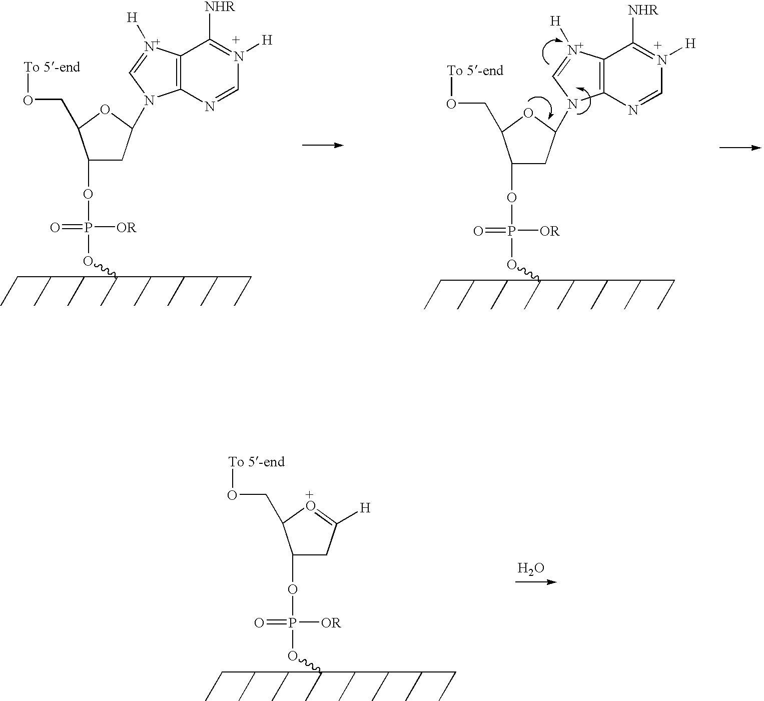 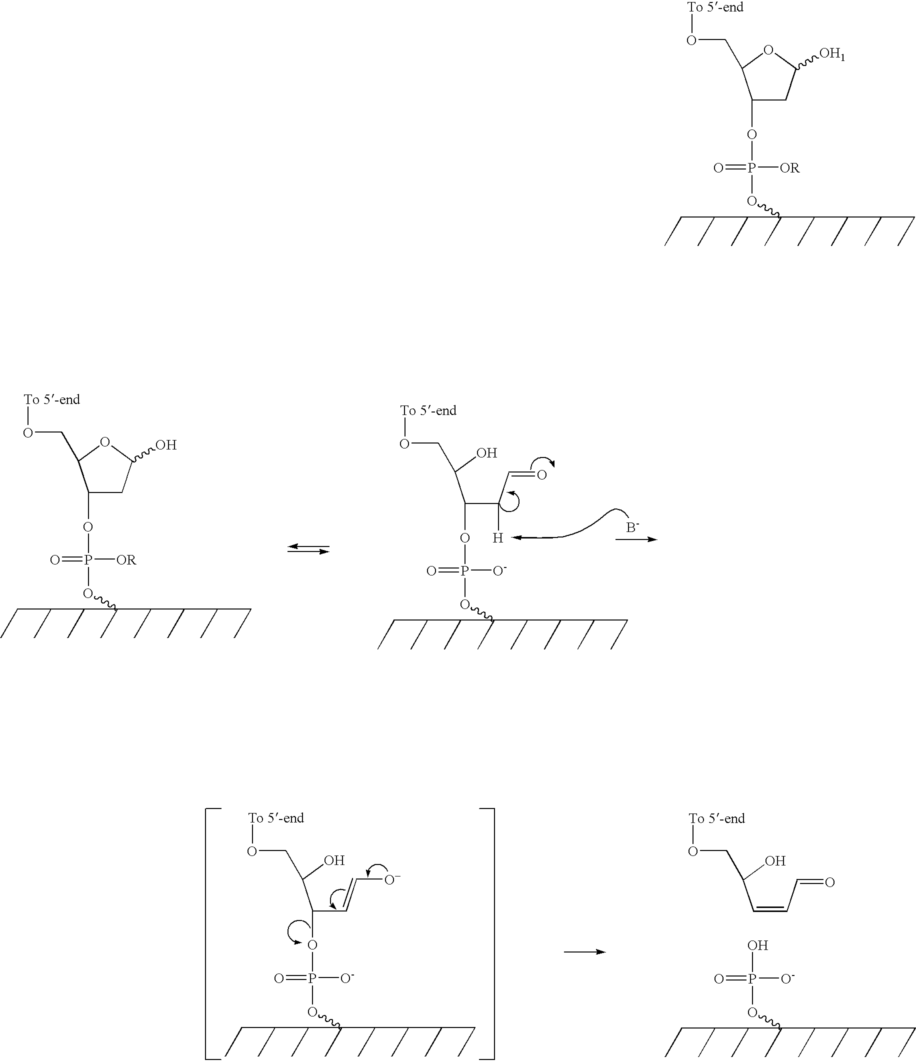
The first line of Scheme 1 shows the desired reaction (deblocking the 5′-hydroxyl at the end of each synthetic cycle) that is responsible for cyclic acid exposure. The second line shows the undesirable, acid-catalyzed side reaction: hydrolysis of the deoxyribose-purine (glycosidic) bond, with conversion of the furan structure of the deoxyribose sugar into an aldose. The base shown in Scheme 1 is adenine (A), because A is by far the more sensitive of the 2 purines. For many embodiments of the application as described below, depurination shall be considered to be strictly a side-reaction of A bases. The final line of Scheme 1 shows the eventual consequences of depurination when the finished oligonucleotide is exposed to a final, base-catalyzed deprotection step to remove protecting groups from the A, C and G bases: the 3′-phosphodiester bond to the aldose sugar is cleaved by βelimination, cleaving the oligonucleotide backbone, with loss of all bases on the 5′-side of the site of depurination.

Depurination of array-bound oligonucleotides is a particularly pernicious problem in those manufacturing protocols where the oligonucleotides on an in situ-synthesized microarray are not subjected to subsequent purification steps meant to retain only full-length products. Thus, depurination during a given synthesis protocol may yield a microarray feature that is both depleted in the intended, full-length oligonucleotide and filled with truncated sequences, where these non-full length sequences at best do nothing and at worst degrade the specificity of the full-length probes.

In view of above described potentially serious impact of depurination on array quality, the quantitative assessment of the degree of depurination is an important component of the overall assessment of microarray quality. As such, there is a need for the development of methods to assess depurination during the in situ manufacture of a nucleic acid array.

### SUMMARY OF THE INVENTION

In situ produced nucleic acid arrays that include at least one depurination probe feature are provided, where the at least one depurination probe feature is made up of in situ produced depurination probes. In using the subject arrays, the arrays are contacted with a nucleic acid sample that includes a target which specifically binds to the full length depurination probe of the depurination feature, and the amount of resultant duplex nucleic acids in the feature is determined (e.g., based on detected signal from the feature) to evaluate the extent of depurination that occurred during in situ synthesis of the array. The subject arrays find use in a variety of different applications, including array fabrication quality control applications, e.g., to determine the extent of depurination in a given lot of nucleic acid arrays produced using an in situ fabrication protocol. Also provided are computer programming, devices that include the same and kits that find use in practicing the subject methods.

### BRIEF DESCRIPTION OF THE DRAWINGS

FIG. 1 provides a depiction of a representative early depurination probe according to an embodiment of the subject invention.

FIG. 2 provides a depiction of a representative late depurination probe according to an embodiment of the subject invention.

FIG. 3 provides a graph of the log of the ratio of late to early signals vs. tether length for a collection of representative late and early depurination probes subjected to different in situ synthesis conditions.

FIG. 4 provides a graph of the ratio of apparent p vs. tether length for a collection of representative late and early depurination probes.

FIG. 5 provides a graph of the log of the signal ratio vs. stagger value obtained for various representative groups of staggered start-depurination probes.

### Definitions

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups. Biopolymers include polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

An “array,” includes any one-dimensional, two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another- and each may-contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein. Also, instead of drop deposition methods, light directed fabrication methods may be used, as are known in the art. Interfeature areas need not be present particularly when the arrays are made by light directed synthesis protocols.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a-hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 nmM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DETAILED DESCRIPTION OF THE INVENTION

In situ produced nucleic acid arrays that include at least one depurination probe feature are provided, where the at least one depurination probe feature is made up of in situ produced depurination probes. In using the subject arrays, the arrays are contacted with a nucleic acid sample that includes a target which specifically binds to the full length depurination probe of the depurination feature, and the amount of resultant duplex nucleic acids in the feature is determined (e.g., based on detected signal from the feature) to evaluate the extent of depurination that occurred during in situ synthesis of the array. The subject arrays find use in a variety of different applications, including array fabrication quality control applications, e.g., to determine the extent of depurination in a given lot of nucleic acid arrays produced using an in situ fabrication protocol. Also provided are computer programming, devices that include the same and kits that find use in practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events which is logically possible, as well as the recited order of events.

All patents and other references cited in this application, are incorporated into this application by reference except insofar as they may conflict with those of the present application (in which case the present application prevails).

As summarized above, the subject invention provides arrays that include at least one depurination probe and methods of using the same, e.g., in evaluating the extent of depurination reactions during in situ array synthesis protocols. In further describing the invention in greater detail than provided in the Summary and as informed by the Background and Definitions provided above, representative embodiments of the subject arrays are described first in greater detail, followed by a review of representative applications of such arrays, e.g., in quality assessment.

Arrays Containing Depurination Probe Features

The subject invention provides nucleic acid arrays that include at least one depurination probe. As summarized above, the subject arrays typically include at least two distinct nucleic acids that differ by monomeric sequence immobilized on e.g., covalently or non-covalently attached to, different and known locations on the substrate surface. Each distinct nucleic acid sequence of the array is typically present as a composition of multiple copies of the polymer on the substrate surface, e.g., as a spot on the surface of the substrate. The number of distinct nucleic acid sequences, and hence spots or similar structures (i.e., array features), present on the array may vary, but is generally at least 2, usually at least about 5 and more usually at least about 10, where the number of different spots on the array may be as a high as about 50, about 100, about 500, about 1000, about 10,000 or higher, depending on the intended use of the array. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 106 or higher, but will generally not exceed about 10⁵ spots/cm². In the subject arrays of nucleic acids, the nucleic acids may be covalently attached to the arrays at any point along the nucleic acid chain, but are generally attached at one of their termini, e.g., the 3′ or 5′ terminus, and typically at their 3′ terminus.

A feature of the subject arrays is that they include at least one depurination probe feature. The number of depurination probe features may vary, but is in certain embodiments less than about 300, such as less than about 100 and and including less than about 70, where the number may be as high as 600 or higher in certain embodiments, but in many embodiments does not exceed about 70.

Each depurination probe feature of the subject arrays is made up of depurination probes, i.e., multiple copies of a given depurination probe or incomplete versions thereof, e.g., due to depurination during synthesis. The total amount of nucleic acid in a given feature may range from about 1.10⁻⁴ pmol to about 0.1 pmol, such as from about 1.10⁻³ pmol to about 1.10⁻³ pmol.

A given full length depurination probe found in a depurination feature of the subject arrays may range in length from about 5 to about 100, such as from about 10 to about 80]-including from about 25 to about 60. The depurination probes are probes that have a known number of purine bases, and specifically Adenosine or A bases. In certain embodiments, the number percent residues of the probes that are A may range from about 20% to about 80%, such as from about 50% to about 70%; where in representative embodiments, the actual number of A residues ranges from about 12 to about 48, including from about 30 to about 42.

In certain embodiments, the depurination probes include two distinct domains, where these domains may be viewed as: (a) a target hybridization domain, which domain is located at the 5′ end of the probe most distant from the surface upon which the probe is immobilized; and (b) a tether domain, which domain is located at the 3′ end of the probe most proximal to the surface upon which the probe is immobilized.

The target hybridization domain, also referred to herein as the hybridizing probe domain, may range in length from about 5 to about 40, including from about 15 to about 30 nt. The hybridization domain is typically heterogenous with respect to the residues which it includes, where in many embodiments the hybridization domain includes all four DNA base residues, i.e., A, G, C and T. The number percent of A residues may vary in this domain, but may range from about 10 to about 40, including from about 20 to about 30 number percent, where the actual number of A residues in representative hybridization domains may range from about 2 to about 10, such as from about 5 to about 7.

The tether domain may range in length from about 0 to about 70 nt, including from about 1 to about 35 nt. The tether domain is typically homogenous with respect to the residues which it includes, where in many embodiments the tether domain is a homogeneous purine domain, and typically a homogeneous A or homo dA domain.

Where the array includes a plurality of different depurination features made up of a plurality of different depurination probes, the target hybridization and tether domains may be the same or different. However, for ease of detection during use, in many such embodiments, the target hybridization domains of the different depurination probes are the same, such that the same labeled target can be used to detect each different depurination probe.

In certain embodiments, the depurination probes can be viewed as early or late probes, depending on the position or layer during the in situ synthesis protocol when their particular synthesis is commenced. For example, where a given in situ synthesis protocol has 60 layers, where each layer is a different activated monomer deposition step, early probes are those probes whose synthesis is commenced near the start of the 60 layer in situ synthesis protocol, e.g., within the first 10 layers, such as within the first 5 layers, including the first layer. In contrast, late probes are commenced at a layer so that the last residue of the late probe is produced near the end of the in situ synthesis cycle, e.g., within about 10 layers of the last layer (such as layer 60), including within about 5 layers of the last layer, such as within about 1 layer of the last layer, including the last layer.

In these embodiments, the total collection or population of depurination probes on a given array may be divided into two subgroups, i.e., early probes and late probes. The number of probes making up a given subgroup may vary, and may range from about 5 to about 100, such as from about 10 to about 80, including from about 20 to about 50. In these embodiments, the numbers of early and late probes are typically substantially even, such that the number ratio in many embodiments of early to late probes may range from about 0.1 to about 10, including from about 0.5 to about 2.

Where the array includes a plurality of different depurination probe features such that the array includes a plurality of different depurination probes, e.g., both early and late probes, the each different member of the plurality or collection of depurination probes may have a different and unique sequence, or alternatively, the constituent members of the population, or subgroups thereof, may have the same sequence but differ from each other only with respect to the particular layer of the overall in situ synthesis protocol in which their synthesis is commenced. As indicated above, where the constituent members of a given population of depurination probes actually differ by sequence, they may at least share a common target hybridization domain, thereby providing for ease of detection during use (e.g., a single labeled target sequence can be used to bind to all of the different constituent members of the depurination probe collection).

In another representative embodiment, the collection of depurination probes present on the array surface can be viewed as a collection staggered start probes (which may also be viewed as layer-tiling probes or overlapping synthesis probes). In these embodiments, all of the staggered start probes have the same sequence and length. The probes may range in length from about 5 to about 50, including from about 15 to about 30 nt. The probes are typically heterogenous with respect to the residues which they include, where in many embodiments the probes include all four DNA base residues, i.e., A, G, C and T. The number percent of A residues may vary in these probes, but may range from about 10 to about 50, including from about 20 to about 30 nt, where the actual number of A residues in representative staggered start depurination probes may range from about 2 to about 13, such as from about 5 to about 8.

While the probes making up a collection of depurination probes are identical nucleic acids, they differ from each other in terms of when their synthesis is commenced during the in situ synthesis protocol. For example, for a given 60 layer in situ synthesis protocol that includes 60 distinct activated monomer deposition steps, each different staggered start depurination probe will have the same length and sequence as all of the other depurination probes, but its synthesis will be commenced at a different layer of the 60 layer protocol. The spacing or number of layers between commencement of any two given probes in a collection of staggered start probes is typically the same, such that the collection of probes has a defined periodicity (in terms of the “skipped” layers between synthesis commencement), where the periodicity may range from about 1 to about 20, including from about 1 to about 5.

Regardless of their particular configuration or structure, depurination probes present on the subject arrays are typically probes whose depurination propensity, i.e., probability of undergoing depurination, during in situ synthesis, may be evaluated or determined based on the nucleotide sequence of the probe. In certain embodiments, depurination susceptibility is evaluated by determining the total “deblock” dose of the depurination probe. By total deblock dose is meant the sum of individual deblock doses over all purines, and particularly over all A nucleotides, in positions of the candidate probe sequence where depurination would markedly affect that probe's hybridization performance. For example, in many embodiments A nucleotides at every position except for that at the 5′-terminus are counted when calculating total deblock dose. In other words, the total deblock dose is the sum of all individual deblock doses for each purine, and in particular each A, residue in the candidate probe sequence, but for the 5′ terminal residue.

In general, any given A residue's individual deblock dose is the total number of deblock cycle exposures experienced by that nucleotide during array manufacture. As such, the general formula for deblock dose d(x) for an A nucleotide written at layer-x of an array made by an in situ synthesis protocol having L total layers is
d(x)=L−x+1 (Eq. A)
Therefore, the overall deblock dose for a sequence containing N A nucleotides written at layers x₁, x₂, . . . , x_{N} during an in situ synthesis protocol is

$$\begin{matrix}{D}_{Total} = {\sum }_{i = 1}^{N} d ( {x}_{i} ) \\ = N ( L + 1 ) - {\sum }_{i = 1}^{N} {x}_{i}\end{matrix} \qquad ( Eq . B )$$

An exemplary algorithm for determining deblock dose is:

Visual Basic code for calculation of deblock dose:

′ Calculate Deblock Dose, with option of omitting 5′-A from calculation,
′ since depurination at this position minimally impacts hyb signal.
′ Sequence is assumed to be provided 5′ to 3′, with 3′ skip (“_”)
′ characters to indicate skipped layers; 5′-skip characters are
′ also permitted, but ignored, since they do not affect deblock dose.

Dim I As Long
Dim N As Long
Dim Noriginal As Long
Dim Acount As Long
Dim aBase As String
DeblockDose2 = 0 ′default
theSequence = UCase(Trim(theSequence)) ′make sequence
unambiguous
N = Len(theSequence)
Noriginal = N
′correct for 5′ skip characters
For I = 1 To Noriginal

If Mid(theSequence, I, 1) = “_” Then

N = N − 1

Else

Exit For

End If

Next I
′ MsgBox “N = ” & N
Acount = 0
If N > tLayers Then

DeblockDose2 = “Illegal Sequence”
Exit Function

End If
For I = 1 To tLayers

If (I <= N And Not omit5PrimeA) Or (I < N) Then

aBase = Mid(theSequence, Noriginal − I + 1, 1)
If aBase = “A” Then Acount = Acount + 1 ′this A
contributes from this layer on

End If
DeblockDose2 = DeblockDose2 + Acount ′add contribution
from this layer

Next I

End Function

Evaluation of Deblock Doses for Early and Late Probes

In those embodiments where the depurination probes present on the array are a collection of early and late probes, the above general formula for determining deblock dose may be modified as described below in order to accommodate for the different layers at which synthesis of the probes is commenced.

Early Probe Deblock Dose:

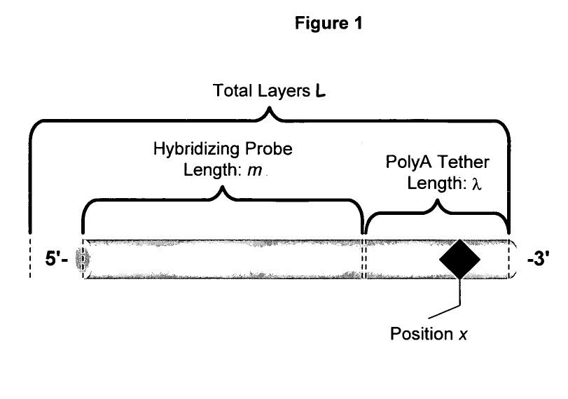

The structure of an early probe is shown in FIG. 1. The deblock dose at any given position x (from the 3′-end) in the tether or probe is given by
e(x)L−x+1 (Eq. 1)
Therefore, the overall tether deblock dose for the tether is

$$\begin{matrix}{E}_{tether} = {\sum }_{x = 1}^{λ} ( L - x - 1 ) \\ = \frac{λ ( 2 L - λ + 1 )}{2}\end{matrix} \qquad ( Eq . 2 )$$

The hybridizing probe deblock dose for a representative probe having a sequence (5′-ATCATCGTAGCTGGTCAGTGTATCC-3′)(SEQ ID NO:01) on the 5-end of an early depurination probe is obtained by summing Eq. 1 over x=λ+4, λ+9, λ+17 and λ+22 (the term for λ+25 is ignored because depurination at that position yields a probe that still hybridizes strongly to its target):
E_{hprobe}=4L−4λ−51 (Eq. 3)
Finally, the overall deblock dose for an early probe is given by
E_{Total}=Eₜₑₜₕₑᵣ+E_{hprobe} (Eq. 4)
Late Probe Deblock Dose:

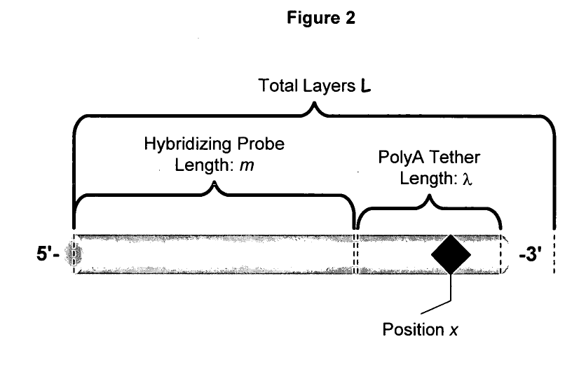

The structure of a late probe is shown in FIG. 2. The deblock dose at any given position x (from the 3′-end) in the tether or probe is given by
λ(x)=m+λ−x+1 (Eq. 5)
The total deblock dose for a late tether is therefore

$$\begin{matrix}{Λ}_{tether} = {\sum }_{x = 1}^{λ} ( m + λ - x + 1 ) \\ = \frac{λ ( 2 m + λ + 1 )}{2}\end{matrix} \qquad ( Eq . 6 )$$

The hybridizing probe deblock dose for same probe sequence of SEQ ID NO:01 on the 5′-end of a late depurination probe is obtained by summing Eq. 5 over x=λ+4, λ+9, λ+17 and λ+22:
Λ_{hprohbe}=4m−51 (Eq. 7)
Finally, the total deblock dose for the late probe is just the sum of the doses for the tether and the hybridization probe:
λ_{Total}=λₜₑₜₕₑᵣ+λ_{hprobe} (Eq. 8)
Staggered Start Probes

As discussed above, another class of probes that can be employed as depurination probes is the class of staggered start probes. As reviewed above, these probes consist of the same probe sequence of length m<L, with synthesis starting at layer s+1, where s is defined as the “stagger” value (s counts the number of skip characters (“_”) that must be placed at the 3′-end of the sequence to cause the writer to delay synthesis initiation until the desired layer). An A nucleotide at position x (from the 3′-end) in a staggered start probe will experience L−x−s+1 exposures to deblock. If the probe contains N A nucleotides at positions x₁, x₂, . . . , x_{N}, then the total deblock dose experienced by the probe is

$$\begin{matrix}Ψ ( s ) = {\sum }_{i = 1}^{N} ( L - {x}_{i} - s + 1 ) \\ = N ( L - s + 1 ) - X , \\ X ≡ {\sum }_{i = 1}^{N} {x}_{i} .\end{matrix} \qquad ( Eq . 22 )$$

The depurination probes of the subject arrays can be positioned at any location on the array. For example, the depurination probes can be positioned in different rows or columns, as convenient.

Utility

In addition to their utility as nucleic acid arrays, reviewed in greater detail below, the subject depurination probe containing arrays find use evaluating or determining, e.g., measuring or quantifying, the extent of depurination in a given in situ array fabrication protocol. In other words, the subject arrays find use in methods of determining the extent of depurination that occurred during a given in situ array synthesis protocol, such as an in situ synthesis manufacturing run.

In these embodiments, following manufacture of an array by an in situ synthesis protocol, the array is contacted under hybridization conditions with a sample that includes nucleic acid target, e.g., labeled target, for the full length dupurination probes, e.g., the hybridizing domain of the depurination probes in a collection of late and early probes. Following sample contact with the array, the array is scanned or read to detect the presence, and typically amount (either relative amount or quantitative amount), of duplex nucleic acids in the one or more depurination features of the array. The presence (and amount) of duplex nucleic acids in the one or more depurination features can be determined using any convenient protocol, e.g., by detecting a signal from the one or more depurination features of the array, and using the detected signal to determine the presence and/or amount of duplex nucleic acid in the feature. (Array hybridizations assays, including labeling and detection protocols, are described in greater detail below).

The detected amount of duplex nucleic acids is then employed to determine the amount of depurination reaction products, i.e., non-full length reaction probes, present in the feature. For example, the amount of detected duplex nucleic acids present in the feature is proportional to the amount of full length probes that are present in the feature, as well as the amount of depurination reaction products present in the feature. More specifically, it is known how many probes would be present in a given feature if no depurination reactions occur, since all of the probes would be full length probes. As such, it is also known how many duplex nucleic acids should be detected following target contact in a feature in which no depurination has occurred. Therefore, from the actual detected amount of duplex nucleic acids in the depurination feature, the number of full length probes, as well as non-full length probes (i.e., depurination reaction products) can readily be determined.

Where the amount of non-full length probes (i.e., depurination reaction products) in a given feature is determined by assessing or detecting a signal from labeled target present in the feature, the resultant signal detected from the one or more depurination features of the array may then be employed to make an evaluation or determination of the extent of depurination that occurred during in situ fabrication of the array. This evaluation may be performed using any convenient protocol that is capable of using signal data from one or more depurination features of the array, where the signal data may be raw or processed, to determine the magnitude of depurination.

The particular protocol employed to determine the magnitude of depurination from the input signal data may vary, e.g., depending on the nature of the depurination probes, the nature of the in situ protocol used to prepare the array, etc. In certain embodiments, the intensity of the detected signal is employed to make a determination of the relative or absolute amount of labeled target that is bound to the feature. This determined value can then be used to determine the amount of full length and the amount of non-full length probes (e.g., depurination side reaction products) in the feature. The determined amount of depurination side reaction products can then be used to assess or evaluate the extent or magnitude of depurination that occurred during synthesis of the array.

One specific representative protocol for determining depurination magnitude from an observed signal of a depurination probe feature includes providing the early and late probes in the list of features included in every QC array used to determine the quality of a manufacturing batch. Those QC arrays are hybridized with target samples prepared in a controlled manner and containing labeled nucleic acid material complementary to the reporter part of the depurination probes. Data analysis of the signals obtained for those depurination probes enable the calculation of apparent depurination yield as described in the Experimental section. Briefly, the log of the ratio of the early probe signal over the late probe signal is first plotted as a function of probe tether length. Then, the obtained curve is fitted to a theoretical model and the apparent depurination yield is derived. For every batch, the apparent depurination yield is compared to values obtained in experiments where the depurination efficiency was modulated (for instance by varying the acid concentration) to estimate the relative quality of the synthesis. Alternatively, the apparent depurination yield can be compared to a control chart in order to estimate the statistical deviation from the controlled process performance. In general, increasing apparent depurination yield will be characteristic of decreased deblock reaction quality while variation in the apparent depurination yield will be characteristic of a drifting process.

As such, once the magnitude of depurination is determined (e.g., in the form of a quantification, either relative or absolute, of the amount of full length and/or depurination side reaction products in a given feature), an evaluation or determination of the extent of depurination that occurred during in situ synthesis of the array can then be made. In other words, the determined magnitude of depurination can be employed to determine the extent of depurination that occurred during synthesis of the array.

The determined magnitude of depurination and therefore extent of depurination that occurred during the in situ fabrication protocol can be employed as a quality control measure, and specifically a depurination quality control measure, of the amount of depurination that occurred during synthesis of the array, and therefore can be employed in the quality evaluation of a lot or batch of arrays produced in a given in situ synthesis run, where the run includes the array displaying the depurination probes. In such applications, the determined magnitude of depurination is compared to a threshold depurination value, where if the determined depurination magnitude does not exceed the threshold value, the array and protocol used to prepare the same, as well as other array members of the lot or batch, are determined as acceptable, at least with respect to the level of depurination produced by the protocol in the member arrays of the lot or batch. Alternatively, if the determined depurination magnitude exceeds a particular threshold depurination value, then the array and protocol used to the prepare the same, as well as other array members of the lot or batch, are determined as unacceptable, at least with respect to the level of depurination produced by the protocol in the member arrays of the lot or batch. In certain embodiments, the depurination threshold can be expressed as the probability of depurination at any given A base on any given cycle. Under these circumstances, the threshold against which the determined value is compared ranges from about 0.3% to about 0.8%, such as from about 0.4% to about 0.6%.

Programming

Programming for practicing at least certain embodiments of the above-described methods is also provided. For example, algorithms that are capable of determining the magnitude of depurination that occurred during a given in situ synthesis protocol from signal values obtained from one or more depurination features are provided. Programming according to the present invention can be recorded on computer readable media, e.g., any medium that can be read and accessed directly or indirectly by a computer. Such media include, but are not limited to: magnetic tape; optical storage such as CD-ROM and DVD; electrical storage media such as RAM and ROM; and hybrids of these categories such as magnetic/optical storage media. One of skill in the art can readily appreciate how any of the presently known computer readable mediums can be used to create a manufacture that includes a recording of the present programming/algorithms for carrying out the above-described methodology.

Additional Utility of Arrays

The subject arrays that include one or more depurination features, as described above, also find use in a variety additional applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g., an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. In certain embodiments, a collection of labeled control targets is typically included in the sample, where the collection may be made up of control targets that are all labeled with the same label or two or more sets that are distinguishably labeled with different labels. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits

Kits for use in analyte detection assays are also provided. The kits at least include the arrays of the invention, as described above. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array based assays.

Kits for use in connection with the depurination quality control applications of the subject invention may also be provided. Such kits preferably include at least a computer readable medium including programming as discussed above and instructions. The instructions may include installation or setup directions. The instructions may include directions for use of the invention.

Providing software and instructions as a kit may serve a number of purposes. The combinations may be packaged and purchased as a means of upgrading an existing fabrication device. Alternatively, the combination may be provided in connection with a new device for fabricating arrays, in which the software may be preloaded on the same. In which case, the instructions will serve as a reference manual (or a part thereof) and the computer readable medium as a backup copy to the preloaded utility.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The following examples are offered by way of illustration and not by way of limitation.

### EXPERIMENTAL

I. Modeling Depurination

The effect of depurination on the intensity profile of a set of depurination probes can be modeled by modeling the separate components of intensity. The general model can be written as
S=S_{B}+Hc_{target}T(λ)Y(λ+m)Q_{intact} (Eq. 9)
where:
S is the observed signal;
S_{B} is the background signal;
H is a constant,
C_{target} is the hybridization target concentration;
T(λ) is the tether enhancement for a tether of length λ,
Y(λ+m) is the full-length oligonucleotide yield after λ+m layers (λfor the tether, m for the hybridizing domain or probe); and
Q_{intact} is the probability that a given depurination probe has not depurinated at any A nucleotide.

The background term is assumed to be small and relatively independent of the depurination probe parameters (i.e., it will be a simple additive constant in the final model). Similarly, the constant H is assumed to be the same for all depurination probes (early or late) on a given array.

Survival Probability (Q_{intact}): The probe survival probability Q_{intact} can be modeled in a straightforward fashion with one assumption: depurination behaves as a pseudo 1st-order reaction. Given this assumption and some standard chemical kinetics, the probability pi that a given A nucleotide depurinates during the i^{th} deblock exposure (which has duration Δtᵢ) is given by
pᵢ=1−e^{-kλt}^{ᵢ}, (Eq. 10)
where k is the pseudo 1^{st}-order rate constant for the depurination reaction. The rate constant k is generally a function of the acid concentration, solvent, temperature, etc.; for the purposes of this description, it is assumed to be the same for all cycles. Note however that depurination rate could depend upon distance from the surface (i.e. it might not be the same for A's in different positions in an oligo). However, the effect of a change in k is exactly the same as the effect of the same percent change in Δtᵢ. Therefore, the model suffers no formal loss of generality, so long as it allows different depurination probabilities pi for different deblock exposures.

The probability that a given A nucleotide survives the ith deblock exposure is simply
qᵢ=1−pᵢ (Eq. 11)
and the probability that a given A at position x survives all of the deblock exposures it experiences is

$$q ( x ) = {\prod }_{all relevant i} {q}_{i} . \qquad ( Eq . 12 )$$

If the probability pᵢ for all A's at all exposures has the same value p for all values of i, then it is easy to show that
q(x)=(1−p)^{d(x)} (Eq. 13)
where d(x) is the deblock dose experienced by the A nucleotide at position x; d(x) is given by Eq. 1 for an early depurination probe or Eq. 5 for a late depurination probe. The overall survival probability Q_{intact} is simply the product over all relevant values of x of the individual survival probabilities q(x):

$$\begin{matrix}{Q}_{intact} = {\prod }_{all relevant x} {( 1 - p )}^{d ( x )} \\ ⇒\end{matrix} \\ \begin{matrix}log ( {Q}_{intact} ) = [ {\sum }_{all relevant x} d ( x ) ] log ( 1 - p ) \\ ≡ {D}_{Total} log ( 1 - p ) \\ ⇒ \\ {Q}_{intact} = {( 1 - p )}^{{D}_{Total}}\end{matrix} \qquad ( Eq . 14 )$$

where Dₜₒₜₐₗ is given by Eq. 4 for an early depurination probe or Eq. 8 for a late depurination probe.

Equation 14 indicates that all of the data from both early and late depurination probes can be modeled together, using Eqs. 9 and 14, provided that we can supply reasonable models of the tether and yield effects. More-importantly, Eq. 14 points the way toward canceling out many of the confounding factors, to produce a pure estimate of depurination.

Tether Effect T(λ): The tether effect appears to arise from a degradation of the binding properties of bases near the array surface. For any given probe, the effect has the general form of a signal that rises from some initial signal S₀ towards some asymptotic signal S_{∞}, with some rate which can be described in various ways. For example, if the tether effect is modeled as a simple association isotherm,

$$S = {S}_{0} + ( {S}_{∞} - {S}_{0} ) \frac{K λ}{K λ + 1} \qquad ( Eq . 15 )$$

where K is a constant that controls the rate of climb of the effect, then it is easy to show that S has climbed halfway from S₀ towards S_{∞} when λ=1/K.

Equation 15 can be used to produce a simple empirical model for the tether effect. Since the general model, Eq. 9, includes a multiplicative constant H, only the shape of the tether effect needs to be modeled. Equation 15 can be rearranged to give

$$\frac{S}{{S}_{∞}} = \frac{{S}_{0}}{{S}_{∞}} + ( 1 - \frac{{S}_{0}}{{S}_{∞}} ) ( \frac{λ / {λ}_{1 / 2}}{1 + λ / {λ}_{1 / 2}} ) \\ ⇒ \\ T ( λ ) = {T}_{0} + ( 1 - {T}_{0} ) ( \frac{λ / {λ}_{1 / 2}}{1 + λ / {λ}_{1 / 2}} ) \qquad ( Eq . 16 )$$

In Eq. 16, λ_{1/2}=1/K and the tether effect has been defined as a surface-dependent depression of target binding, i.e. the binding of a tetherless probe is decreased by a factor T₀<1. This multiplier increases towards 1 as λ→∞; half of the increase has occurred when λ=λ_{1/2}.

Synthetic Yield Y(λ+m): The synthetic yield is usually modeled as a simple average stepwise yield y raised to the power of the number of synthetic steps, in this case λ+m. The chief problem with modeling the synthetic yield is that it has a functional form quite similar to that of the depurination survival probability Q_{intact} (i.e. a positive number<1 raised to a power that depends upon the number of synthetic steps). Therefore, yield effects are potentially confounded with depurination effects. However, as will be shown below, the existence of two types of depurination probes (early and late) enables the cancellation of the yield effects (as well as several other effects). Thus, proper analysis of the data offers a straightforward route to calculation of the single-step depurination probability p.

Analysis and Predictions: Equations 9 and 14 can be combined to make a testable prediction which, if correct, also provides a straightforward method for estimating p, the probability of depurination of a given A nucleotide during a single deblock cycle. According to Eq. 9, in light of Eq. 14, the ratio of the signals from early and late depurination probes of the same tether length λ is given by

$$\frac{{S}_{late} ( λ )}{{S}_{early} ( λ )} = \frac{{S}_{B} + H {c}_{target} T ( λ ) Y ( λ + m ) {( 1 - p )}^{{Λ}_{Total}}}{{S}_{B} + H {c}_{target} T ( λ ) Y ( λ + m ) {( 1 - p )}^{{E}_{Total}}} \qquad ( Eq . 17 )$$

If net signals are used, and the background subtraction is accurate,

$$\begin{matrix}\frac{{S}_{late} ( λ )}{{S}_{early} ( λ )} ≅ \frac{H {c}_{target} T ( λ ) Y ( λ + m ) {( 1 - p )}^{{Λ}_{Total}}}{H {c}_{target} T ( λ ) Y ( λ + m ) {( 1 - p )}^{{E}_{Total}}} \\ = \frac{{( 1 - p )}^{{Λ}_{Total}}}{{( 1 - p )}^{{E}_{Total}}}\end{matrix} \qquad ( Eq . 18 )$$

Taking the log of both sides of Eq. 18, then substituting from Eqs. 2, 3, 4, 6, 7 and 8 yields

$$\begin{matrix}log [ \frac{{S}_{late} ( λ )}{{S}_{early} ( λ )} ] = ( {Λ}_{Total} - {E}_{Total} ) log ( 1 - p ) \\ = [ \begin{matrix}\frac{λ ( 2 m + λ + 1 )}{2} + 4 m - 51 - \\ \frac{λ ( 2 L - λ + 1 )}{2} - 4 L + 4 λ + 51\end{matrix} ] log ( 1 - p ) \\ = [ ( λ + 4 ) ( λ + m - L ) ] log ( 1 - p )\end{matrix} \qquad ( Eq . 19 )$$

Note that the ratio of the late probe signal to the early probe signal is expected to be ≧1, with equality when λ+m=L (i.e. early and late probes are exactly the same, since the total probe length is L). The log ratio should therefore be ≧0 with equality when λ+m=L. Since (1−p)<1, its log is <0; therefore, to be consistent, its multiplier must also be negative (or zero when λ+m=L). It is clear from FIGS. 1 and 2 that L≧λ+m. In addition, it is clear that Eq. 19 yields a log ratio of zero when λ+m=L. Therefore, Eq. 19 passes two simple consistency checks. Equation 19 makes a very powerful prediction: a plot of the log ratio of late to early probe signals versus tether length λ can be fit by a simple quadratic form in λ, whose sole adjustable parameter is p, the probability that a single A nucleotide depurinates during a single deblock exposure. Alternatively, Eq. 19 can be rewritten as

$$[ \frac{1}{( λ + 4 ) ( λ + m - L )} ] log [ \frac{{S}_{late} ( λ )}{{S}_{early} ( λ )} ] = log ( 1 - p ) \qquad ( Eq . 20 )$$

In other words, a plot of the left hand side of Eq. 20 as a function of tether length λ should yield a flat line with average value log(1-p). In addition, Eqs. 19 and 20 make predictions about the effects of varying deblock times: by Eq. 10,

$$\begin{matrix}log ( 1 - p ) = log [ 1 - ( 1 - {ⅇ}^{- k Δ t} ) ] \\ = log ( {ⅇ}^{- k Δ t} ) \\ = \frac{- k Δ t}{ln ( 10 )}\end{matrix} \qquad ( Eq . 21 )$$

Thus, Equation 21 predicts that any quantity that depends linearly upon log(1-p) will depend linearly upon deblock time Δt.
II. Validation of Model

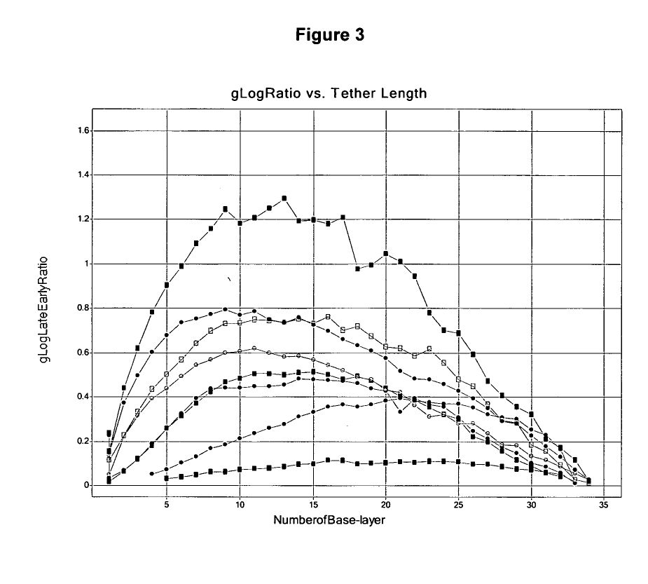

Initial Tests: Equation 19 predicts that a plot of the log (late:early) ratio as a function of the tether length λ should be parabolic (i.e. quadratic in λ). A test of this hypothesis is shown in FIG. 3, using depurination probes manufactured using deblock times of 10 sec. (red), 60 sec. (blue), 120 sec. (yellow) and 240 sec (black). At each deblock time tested, 2 slides were analyzed.

The expected shape is a parabola with a maximum at %=15.5 (To show this, differentiate Eq. 19 with respect to λ and set the result=0). The value of the log ratio at the maximum should be −380.25*log(1−p). FIG. 3 shows data from slides 2 (circles) and 1 (squares), for deblock times of 10 sec; (red), 60 sec. (blue), 120 sec. (yellow) and 240 sec (black).

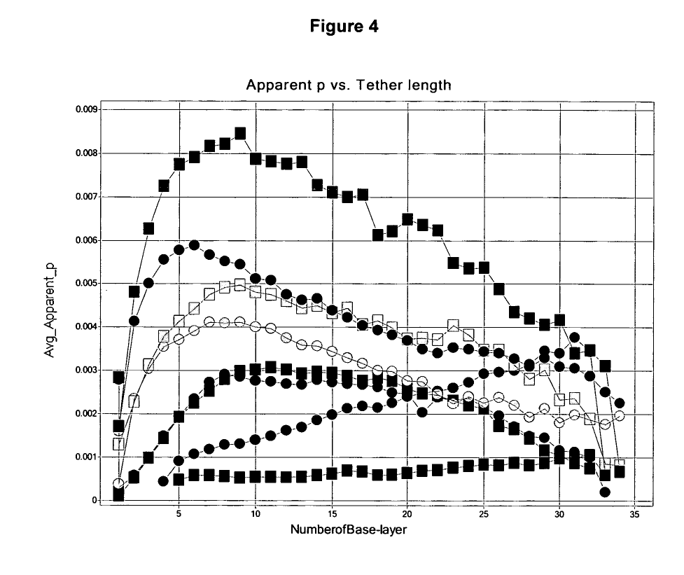

From FIG. 3, it is apparent that the model has the correct general form (the data are parabolic or nearly parabolic, and the maximum increases as deblock time increases). However, the real data show additional complications: the shapes are not pure parabolas (they show some asymmetry) and do not always peak at the expected position. This may be indicative of additional, unmodeled effects (e.g. coupling yields or depurination probabilities that vary with layer). The same data can be used to calculate apparent values of p as a function of λ, via Eq. 20. The results of this calculation are shown in FIG. 4. The color and shape legends for FIG. 4 are as in FIG. 3. The maximum values of p vary from a low of <0.001 (10 sec deblock) to a high of 0.008 (240 sec deblock). However, the profiles are not flat as a function of λ, again indicating that the model has not captured all phenomena.

IV. Modeling Depurination of Staggered Start Probes

As described earlier, another class of probes that shows evidence of the effects of depurination are staggered start probes. These probes consist of the same probe sequence of length m<L, with synthesis starting at layer s+1, where s is defined as the “stagger” value. An A nucleotide at position x (from the 3′-end) in a staggered start probe will experience L−x−s+1 exposures to deblock. If the probe contains N A nucleotides at positions x₁, x₂, . . . , x_{N}, then the total deblock dose experienced by the probe is

$$\begin{matrix}Ψ ( s ) = {\sum }_{i = 1}^{N} ( L - {x}_{i} - s + 1 ) \\ = N ( L - s + 1 ) - X , \\ X ≡ {\sum }_{i = 1}^{N} {x}_{i} .\end{matrix} \qquad ( Eq . 22 )$$

By analogy to the derivation of Eqs. 17-19, we may then write

$$\begin{matrix}log [ \frac{S ( {s}^{′} )}{S ( s )} ] = [ Ψ ( {s}^{′} ) - Ψ ( s ) ] log ( 1 - p ) \\ = N ( s - {s}^{′} ) log ( 1 - p ) .\end{matrix} \qquad ( Eq . 23 )$$

For example, for standard 24-mer or 25-mer staggered start probes referenced to the s=1 probe, Eq. 23 becomes

$$log [ \frac{S ( s )}{S ( 1 )} ] = - 4 ( s - 1 ) log ( 1 - p ) . \qquad ( Eq . 24 )$$

V. Validation of Staggered Start Probe Model

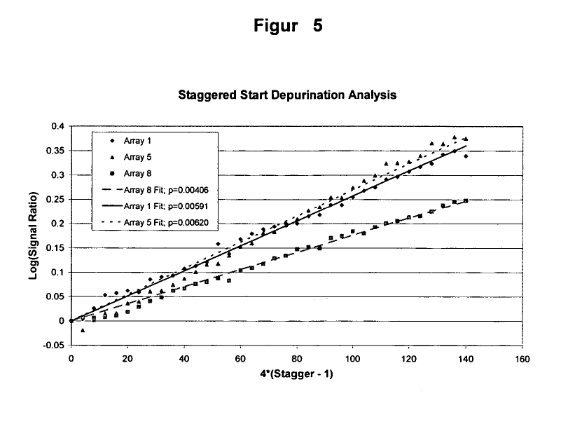

As a test, embedded QC staggered start profiles have been analyzed according to Eq. 24. Three representative plots (along with calculated values of p) are shown in FIG. 5.

It is clear from FIG. 5 that this analysis of the data works well: the log ratio data yield good linear fits, and the slopes translate into sensible values of the per-layer-per-A depurination probability p. In fact, the estimates of p are in the same range seen in the previous section, during analysis of depurination probes. Thus, it appears that the two methods are measuring the same phenomenon.

VI. Use of Depurination Probability

The apparent depurination yield obtained from the depurination probes and the depurination probability obtained from the staggered start probes may be used to assess the contribution of the deblock reaction to the synthesis quality of a microarray manufacturing batch. In a first method, several processes may be relatively compared by comparing the apparent depurination yield obtained for each processes. For instance, the impact of temperature, reagent composition, reaction time, and/or any other factor may be quantified relatively to a control process. The deblock yield may then be used to tune a process into a desired performance range. In a second method, the stability of a process may be controlled in time by generating a control chart of all apparent depurination yield obtained. The curve will be characterized by a mean value and standard deviation. In time, the stability of the process may be controlled by evaluating the apparent depurination yields of future arrays. For example, apparent depurination yields that are within 3 standard deviation of the mean value of the process may be found “in control”. Apparent depurination yields deviating from this range will indicate a drift in the process performance.

It is evident from the above discussion that the above-described invention provides methods for the ready determination of the extent of depurination that occurs during a given in situ synthesis protocol, and is well suited for deployment as a general method of routinely monitoring depurination in a production setting. As such, the subject invention represents a significant contribution to the art.

As reviewed above, the subject invention provides methods of identifying the extent of depurination during the synthesis of nucleic acid arrays. However, the subject invention can be used with a number of different types of arrays in which a plurality of distinct polymeric binding agents (i.e., of differing sequence) are stably associated with (i.e., immobilized on) at least one surface of a substrate or solid support by a step-wise synthesis protocol. As such, the polymeric binding agents may vary widely, however polymeric binding agents of particular interest include peptides, proteins, nucleic acids, polysaccharides, synthetic mimetics of such biopolymeric binding agents, etc. In many embodiments of interest, the biopolymeric arrays are arrays of nucleic acids, including oligonucleotides, polynucleotides, cDNAs, mRNAs, synthetic mimetics thereof, and the like.

As such, while the subject methods and devices find use in producing nucleic acid arrays (as described above), the subject devices also find use in the production of non-nucleic acid ligand arrays in which a step-wise or in situ synthesis approach is employed. That is, any of a number of different types of ligand arrays may be produced by the methods of the subject invention, where a first member of a binding pair, typically referred to herein as the ligand is stably associated with the surface of a substrate. For ease of description only, the subject methods and devices described above were described primarily in reference to nucleic acid arrays, where such examples are not intended to limit the scope of the invention. It will be appreciated by those of skill in the art that the subject devices and methods may be employed for use in the production of other types of ligand arrays, e.g., peptide arrays etc., where the ligands of arrays may be synthesized using a step-wise synthesis protocol, particularly where a degradation side reaction may occur in the employed step-wise synthesis protocol.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of detecting the presence of depurination reaction products on a surface of an in situ produced nucleic acid array, said method comprising:
(a) contacting an in situ produced nucleic acid array that includes at least one depurination probe feature of a depurination probe with a sample comprising a target nucleic acid that specifically binds to said depurination probe; and
(b) detecting the amount of resultant binding complexes in said depurination probe feature to determine the presence of depurination reaction products on said surface.

2. The method according to claim 1, wherein said method is a method of determining the amount of depurination reaction products on said surface.

3. The method according to claim 2, wherein said amount is a relative amount.

4. The method according to claim 1, wherein said target nucleic acid is labeled and said detecting comprising detecting a signal from said depurination probe feature.

5. The method according to claim 4, wherein said label is fluorescent and said signal is a fluorescent signal.

6. The method according to claim 5, wherein said fluorescent signal has an intensity that is inversely proportional to the amount of depurination reaction products in said depurination probe feature.

7. The method according to claim 1, wherein said array includes two or more different depurination probe features each corresponding to a distinct depurination probe.

8. The method according to claim 7, wherein said array includes at least one early depurination probe feature and at least one late depurination probe feature.

9. The method according to claim 1, wherein said array includes two or more identical depurination probe features whose synthesis was started at different times.

10. The method according to claim 1, wherein said depurination probe has a known deblock dose.

11. The method according to claim 1, wherein said method further comprises evaluating the level of depurination that occurred during in situ fabrication of said array.

12. The method according to claim 11, wherein said method is a method of evaluating the quality of an in situ nucleic acid array synthesis fabrication protocol.

13. The method according to claim 12, wherein said method is employed to evaluate the quality of a plurality of nucleic acid arrays fabricated according to said protocol.

14. An array comprising a set of two or more nucleic acid depurination features.

15. The array according to claim 14, wherein each member of said set comprises probes having identical probe hybridization domains and different tether domains.

16. The array according to claim 15, wherein said different tether domains are polyA domains of differing length.

17. The array according to claim 16, wherein said polyA domains range from about 1 to about 35 nt in length.

18. The array according to claim 14, wherein said nucleic acid depurination probes of said set are immobilized on a surface of a solid support.

19. The array according to claim 14, wherein said set includes both early and late depurination features.

20. The array according to claim 14, wherein said set comprises a collection of staggered start depurination probes.

21. A method of detecting the presence of a nucleic acid analyte in a sample, said method comprising:
(a) contacting a nucleic acid array according to claim 14 having a nucleic acid ligand that specifically binds to said nucleic acid analyte with a sample suspected of comprising said analyte under conditions sufficient for binding of said analyte to said nucleic acid ligand on said array to occur; and
(b) detecting the presence of binding complexes on the surface of said array to detect the presence of said analyte in said sample.

22. The method according to claim 21, wherein said sample comprises a collection of labeled target nucleic acids that specifically bind to said depurination probes nucleic acids.

23. A method comprising transmitting a result from a reading of an array according to the method of claim 21 from a first location to a second location.

24. The method according to claim 23, wherein said second location is a remote location.

25. A method comprising receiving a transmitted result of a reading of an array obtained according to the method claim 21.

26. A kit for use in a nucleic acid analyte detection assay, said kit comprising:
an array according to claim 14.

27. The kit according to claim 26, wherein said kit further comprises labeled target nucleic acids that specifically bind to said depurination probe nucleic acids.

28. A computer-readable medium having recorded thereon a program that determines the presence of depurination reaction products in a nucleic acid array from a signal observed from a depurination probe feature of said array.
